(19) United States
(12) Patent Application Publication
(10) Pub. No.: US 2021/0393500 A1
(43) Pub. Date: Dec. 23, 2021

(54) HAIR TREATMENT COMPOSITION, METHODS AND USES THEREOF

(71) Applicant: UNIVERSIDADE DO MINHO, Braga (PT)

(72) Inventors: Artur Manuel CAVACO PAULO, Braga (PT); Maria Madalena ALVES MARTINS DE SÁ, Braga (PT)

(21) Appl. No.: 17/266,853

(22) PCT Filed: Aug. 9, 2019
(86) PCT No.: PCT/IB2019/056805

§ 371 Date: Feb. 8, 2021

(30) Foreign Application Priority Data
Aug. 9, 2018 (PT) 111107

(51) Int. Cl. A61K 8/64; A61K 8/44; A61K 8/41; A61K 8/49; A61K 8/42; A61K 8/73; A61Q 5/12

(52) U.S. Cl. CPC A61K 8/64; A61K 8/447; A61K 8/416; A61Q 5/12; A61K 8/42; A61K 8/731; A61K 8/4946

(57) ABSTRACT

The present disclosure relates to a composition comprising a general ionic liquid, a eutectic mixture or a deep eutectic mixture as a solvent and at least one soluble auxiliary substance. The auxiliary substance might be a peptide or reducing agent or a surfactant or cosmetic component or a combination of them, with the purpose to modify the characteristics of human hair.

### TECHNICAL FIELD

The present disclosure relates to a composition comprising a general ionic liquid, a eutectic mixture or a deep eutectic mixture as a solvent and at least one soluble auxiliary substance. The auxiliary substance might be a peptide or reducing agent or a surfactant or cosmetic component or a combination of them, with the purpose to modify the characteristics of human hair.

### BACKGROUND

Ionic liquids are liquids composed of ions, an organic cation and an anion that can be organic or inorganic. They are composed of charged units, hence they present low vapor pressures and are considered non-volatile [1]. Eutectic liquids are known as DES (deep eutectic solvents) and are the new class of ionic liquids since they are analogous, sharing many characteristics and properties. In deep eutectic liquids, the boiling point of the mixture is relative lower (generally bellow room temperature) than their constituents.

Deep eutectic liquids exhibit a low or negligible vapor pressure, relatively wide liquid range, good solvation properties and non-flammability. They present ability to customize properties as constituents ratio, temperature and constituents nature [2]. Eutectic solvents contain large, non-symmetric ions with low lattice energy and consequently low melting points. Typically, there are three types of eutectic solvents: 1) complexation of a quaternary ammonium salts, 2) metals into formulations and 3) based on hydrogen bond donor (HBD) or hydrogen bond acceptor (HBA).

Common chemical hair processes as straightening or perming style make permanent changes to the hair cortex, destroying parts of its structure. These processes tend to weaken and dry hair fiber making it brittle. As a result of the hair damage, the fiber loses some of its properties as strength and elasticity [3,4]. The commercial products essentially based on a strong alkali, ammonium thioglycolate, in a form of hair masks or serums and then a neutralizing solution it is used to replace the pH back to normal, usually acidic solution in a form of shampoo or conditioner.

Minimize hair damage to manipulate the change of the hair shape it's the driving force for haircare industry. The solutions here proposed can have a high impact on how human beings change the shape of their hair using green solutions without damaging their health. Eutectic and ionic solvents have several advantages including no need of purification, non-toxicity, biocompatibility, biodegradability, and can be considered as environmentally benign solvents.

These facts are disclosed in order to illustrate the technical problem addressed by the present disclosure.

### GENERAL DESCRIPTION

The proposed alternative green solution replaces harsh reducing alkaline agents with benign environment solvents such as ionic and eutectic liquids that act at mild conditions to change the characteristics of human hair. This green solution is therefore expected to have a high impact on the haircare cosmetic industry with direct benefits on the environment and on humans. This approach present lowers side effects, easy application and lower cost. Our solution represents a novel and green method of using peptide sequences (described at WO2015056216) [5] derived from hair proteins (human hair keratins and keratin associated proteins—KAP) and/or natural reducing agents such as cysteine, lysine-cysteine-leucine, lysine-cysteine-cysteine-leucine, lysine-cysteine-leucine-OEt, lysine-cysteine-cysteine-leucine-OEt, or chemical reducing agents or any other auxiliary component or a combination of them, incorporated into the ionic or eutectic liquid. The mixture remains liquid at room temperature despite having little viscosity. Once applied onto hair fiber, the mixture promotes hair fibre swelling and facilitate better diffusion of the auxiliary agent. The auxiliary agent is incorporated into the hair fibre thus leading to novel fibre characteristics such as coloring, softening and/or rearrangement of intra and inter molecular disulphide bonds (leading to hair shape changes).

The hair treatment composition of the present disclosure comprises at least a green solvent, namely a eutectic liquid, a deep eutectic liquid or an ionic liquid which is a greener alternative to many components in hair cosmetic compositions. The present disclosure describes a solution composition comprising a green solvent (either eutectic liquids or ionic liquids) and auxiliary substances. The hair treatment composition of the present disclosure has a synergistic effect, the composition can swell human hair fibres and can diffuse into the human hair changing characteristics of hair fibres.

This composition may be used for treatment of human hair, animal hair or animal fur for hair strengthening agent, hair softening agent, hair curling, staining agent, anti-humidity agent, hair dye for hair coloring, hair anti-frizz agent, or as a hair conditioning agent.

In an embodiment, the hair treatment composition of the present disclosure can be applied using the individual solid components on the hair (human or animal) to be treated, meaning that the application can be done in two forms:
make the composition and after applying to the hair or;
apply all the reagents onto the hair to be treated, mixture the hair and the reagents, heat and allow the treatment composition to stay on the hair for some minutes.

In the present disclosure, hair includes human hair, animal hair and animal fur.

An aspect of the present subject-matter discloses a hair treatment composition comprising a solvent selected from a list consisting of an ionic liquid (“ILs”), a eutectic mixture and combinations thereof; and an auxiliary component selected from a list consisting of: reducing agent, adjuvant, and mixtures thereof.

A eutectic liquid or eutectic mixture can be defined as a mixture of two or more components which usually do not interact to form a new chemical compound. Eutectic mixture formation is usually governed by the following factors: (a) the components must be miscible in liquid state and mostly immiscible in solid state, (b) intimate contact between eutectic forming materials is necessary for contact induced melting point depression, (c) the components should have chemical groups that can interact to form physical bonds such has intermolecular hydrogen bonding etc., (d) the molecules which are in accordance with modified VantHoff's equation can form eutectic mixtures.

Ionic liquids (“ILs”) are organic salts that are liquid at temperatures below 100° C. These ILs have received considerable attention as substitutes for volatile organic solvents. Since they are non-flammable, non-volatile and are recyclable, they are classified as green solvents. Due to their solvating potential, thermal stability, and tunable properties (by selecting suitable cations and anions), they are considered favorable medium for chemical syntheses.

It is also disclosed a hair treatment composition comprising: a solvent; and an auxiliary agent; wherein the solvent is selected from the following: an ionic liquid, a eutectic mixture, a deep eutectic mixture, or combinations thereof; wherein the auxiliary agent is selected from the following: an adjuvant, a reducing agent, a synthetic peptide with a sequence length of from 6 to 12 amino acids where 2-5 of the amino acids are cysteines, or mixtures thereof.

In an embodiment, the concentration of auxiliary agent varies from 0.01% to 20% (weight by weight).

In an embodiment, the concentration of auxiliary agent varies from 1% to- 6% (weight by weight).

In an embodiment, the composition may further comprise an adjuvant, a peptide with a sequence length of 6-12 amino acids, where 2-5 of those amino acids are cysteines, and mixtures thereof.

In an embodiment, at least one peptide is selected from the following list with a degree of identity of at least 90%: SEQ.ID No. 1, SEQ.ID No. 2, SEQ.ID No. 3, SEQ.ID No. 4, SEQ.ID No. 5, SEQ.ID No. 6, SEQ.ID No. 7, SEQ.ID No. 8, SEQ.ID No. 9, SEQ.ID No. 10, SEQ.ID No. 11, SEQ.ID No. 12, SEQ.ID No. 13, SEQ.ID No. 14, SEQ.ID No. 15, SEQ.ID No. 16; preferably SEQ.ID No. 4 and/or SEQ.ID No. 16. Preferably with a degree of identity of at least 95%, 96%, 97%, 98%, or 99%.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Global percentages of similarity and identity may also be determined using one of the methods available in the MatGAT software package (Campanella et al., BMC Bioinformatics. 2003 Jul. 10; 4:29. MatGAT is an application that generates similarity/identity matrices using protein or DNA sequences). Minor manual editing may be performed to optimise the alignment between conserved motifs, as would be apparent to a person skilled in the art. The sequence identity values which are indicated in the present subject matter as a percentage were determined over the entire amino acid sequence using BLAST with the default parameters.

In an embodiment, at least one peptide is selected from following list: SEQ.ID No. 1, SEQ.ID No. 2, SEQ.ID No. 3, SEQ.ID No. 4, SEQ.ID No. 5, SEQ.ID No. 6, SEQ.ID No. 7, SEQ.ID No. 8, SEQ.ID No. 9, SEQ.ID No. 10, SEQ.ID No. 11, SEQ.ID No. 12, SEQ.ID No. 13, SEQ.ID No. 14, SEQ.ID No. 15, SEQ.ID No. 16; preferably SEQ.ID No. 4 and/or SEQ.ID No. 16.

In an embodiment, the solvent amount is from 1 to 100,000 mmol, preferably 10-50,000; more preferably 500-10,000.

In an embodiment, the solvent concentration in the composition varies from 0.1-0.9% (wt/wt); preferably 0.15-0.85% (wt/wt); more preferably 0.7-0.3% (wt/wt).

In an embodiment, the ionic liquid is selected from a list consisting of: 1-butyl-3-methylimidazolium acetate with N,N-dimethylacetamide; 1-Butyl-3-methylimidazolium cysteine with 1-Butyl-3-methylimidazolium hydroxide with cysteine; (2-hydroxyethyl)trimethylammonium with amino acid glycinate or cysteine and Cholinehydroxide with amino acid; Choline thioglycolate; 1-allyl-3-methylmidazolium dicyanamide; 1-Allyl-3-methylimidazolium chloride; 1-butyl-3-methylimidazolium chloride ionic liquid; or mixtures thereof.

In an embodiment, the eutectic liquid is selected from a list consisting of: Choline chloride-urea; Decanoic acid (DecA)-tetraoctylammonium; chloride; Malonic acid-choline chloride; Oxalic acid-choline chloride; Choline chloride: ethanolamine-based; Tryptophan fluoborate (TrpBF4)/urea; Urea-Glucose-Citric Acid; Urea-Glucose-Fructose; Urea-Tartaric Acid; Urea-Choline chloride; Glucose-Fructose-Sorbitol; Citric Acid-Fructose; Glucose-Citric Acid-Water; Tartaric Acid-Fructose; Proline-Glutamic Acid; Proline-Glutamic Acid; Proline-Oxalic Acid; Proline-Tartaric Acid; Ornitine-Tartaric Acid; Arginine-Tartaric Acid; Citrulline-Tartaric Acid; Arginine-Oxalic Acid; Proline-Malic Acid; Arginin-Malic Acid; Ornitine-Malic Acid; Citrulline-Malic Acid; Proline-Citric Acid; Arginine-Citric Acid; Ornitine-Citric Acid; Citrulline-Citric Acid; Proline-Glucose; Proline-Fructose; Proline-Choline Chloride; Choline Chloride-Malic Acid; Malic acid-glucose; Choline chloride-glucose; Adipic acid-choline chloride; Benzoic acid: choline chloride; Phenylacetic acid-choline chloride; Phenylpropionic acid-choline chloride; Succinic acid-choline chloride; Glycerol-choline chloride; Glucose-malic acid; Fructose-malic acid; Sucrose-malic acid; Glucose-citric acid; Sucrose-citric acid; Trehalose-citric acid; Thiourea choline chloride; Acetamine choline chloride; Benamide choline chloride; or mixtures thereof.

In an embodiment, the reducing agents are selected from a list consisting the following: peptide KCL; peptide KCCL; peptide KCL-OEt; peptide KCCL-OEt; Cysteine amino acid; Dithiothreitol; Sodium bisulphate; 2-mercaptoethanol; Thioglycolic acid; Urea; or mixtures thereof.

In an embodiment, the adjuvants are selected from the following list: carbohydrate, polysaccharide, modified cellulose, cellulose, chitosan, dimethyl sulfoxide, organic polymer, humectant, oils, natural polymer derived, humectant, silicone, protein, emollient ester, alkanolamide, amine, salt, aliphatic alcohol, amine oxide, chelate, fatty acid, PEG material, polymer, alcohol, or mixtures thereof.

In an embodiment, the composition may further comprise, comprising softener, dye, pigment, fragrance, surfactant, emulsifier, preservative, thickener vitamin, buffer, antimicrobial agent, antibacterial agent, disinfectants agents, emulsifier, preservative, UV filter, anti-static agent, pigment, tensioactive, or mixtures thereof.

In an embodiment, the surfactant is selected from the following list: anionic surfactant, amphoteric surfactant, cationic surfactant, or mixtures thereof.

In an embodiment, the softener is cationic softeners such as quaternary ammonium salts, amine salts, imidazolines and quaternaries with ester, organic oil.

In an embodiment, the composition may be used for hair strengthening agent, hair softening agent, hair curling agent, hair anti-frizz agent, hair anti-humidity agent, protectant for coloured hair, dye for hair colouring, hair volumizing agent, staining agent, or hair conditioning agent.

In an embodiment, the composition may be used for hair curling agent, a hair volumizing agent, or a hair conditioning agent.

### BRIEF DESCRIPTION OF THE DRAWINGS

The following figures provide preferred embodiments for illustrating the disclosure and should not be seen as limiting the scope of invention.

FIG. 1: Schematic representation of a Caucasian hair sample after coloration with eutectic liquid with Basic Red 2.

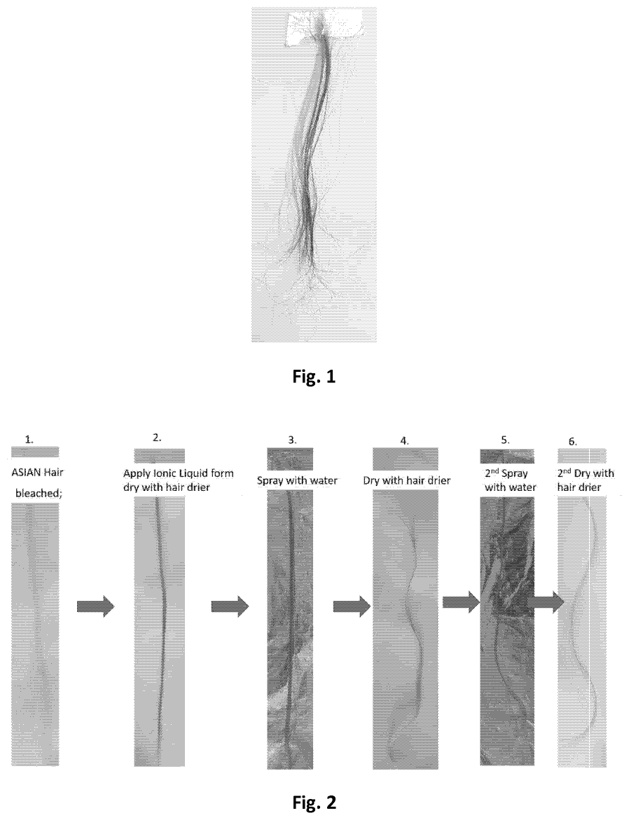

FIG. 2: Induced waving of Asian hair treated by [BMIM]Cl-DMSO-cellulose, in two cycles wet (spray with water)-dry (bow-dry). Length variation of first cycle about 13% and after second cycle 16%. DMSO—Dimethyl sulfoxide, [BMIM]Cl-1-butyl-3-methylimidazolium chloride.

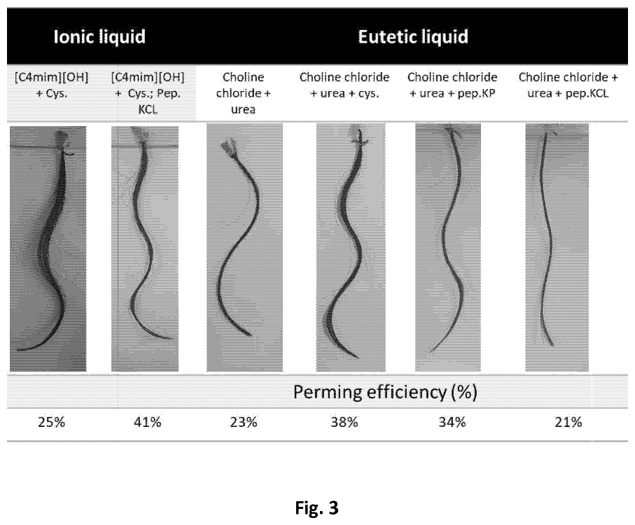

FIG. 3: Schematic representation of hair sample treated with:
Ionic Liquid and reducing agent: 1-Butyl-3-methylimidazolium hydroxide and Cysteine;
Ionic Liquid and reducing agent: 1-Butyl-3-methylimidazolium hydroxide and Cysteine and lysine-cysteine-leucine peptide;
Eutetic mixture and reducing agent: choline chloride+urea—an eutectic solution for comparative purpose;
Eutetic mixture and reducing agent: choline chloride+urea+cysteine;
Eutetic mixture and reducing agent: choline chloride+urea+synthetic peptide—Pep KP (SEQ ID 16).
Eutetic mixture and reducing agent: choline chloride+urea+lysine-cysteine-leucine peptide.

### DETAILED DESCRIPTION

The present disclosure relates to a composition comprising deep eutectic or general ionic liquids and at least one soluble auxiliary substance. The auxiliary substance might be a peptide or reducing agent or cosmetic component or synthetic peptide or a combination of them, with the purpose to modify the characteristics of human hair.

In an embodiment, the deep euthetic mixtures (or euthetic liquids) are characterized by being generally soluble at room temperatures (namely 20° C.) when their individual components are solid (for example the molar mixture 2:1 of choline chloride and urea have a melting point of is 12° C. while the individual component are 302° C. and 133° C. respectively). Ionics liquid are mixture high molecular weight ions and cations which have normally low vapor pressure. To this mixture auxiliary agents can be added (tables 2-4). Auxiliary agents can be selected among peptides (table 2), reducing agents (table 3) or other components (table 4), or their mixtures.

In an embodiment, protein keratin and keratin-associated proteins (KAPs) have high sulfur content present in cysteine amino acid residue. The presence of sulfur it is very important in the stability of hair structure once it allow the formation of intra- and inter-disulphide bonds between amino acids of the polypeptide chains.

In an embodiment, the current disclosure uses synthetic peptide sequences analogous to keratin proteins described in patent document WO/2015/056216, as well as peptides with and without an ethyl ester group (KCL-OEt, KCL, KCCL-OEt, KCCL) which can be used as reducing agents (Table 3). The peptide sequences are described by one letter code of amino acids. The code is as follows in Table 1.

TABLE 1
List of amino acid letter code and the respective name.

| Amino acid one letter code | Amino acid name |
|---|---|
| H | Histidine |
| R | Arginine |
| K | Lysine |
| I | Isoleucine |
| F | Phenylalanine |
| L | Leucine |
| W | Tryptophan |
| A | Alanine |
| M | Methionine |
| P | Proline |
| V | Valine |
| C | Cysteine |
| N | Asparagine |
| G | Glycine |
| S | Serine |
| Q | Glutamine |
| Y | Tyrosine |
| T | Threonine |
| D | Aspartic acid |
| E | Glutamic acid |

TABLE 2
List of peptide sequences, with a degree
of identity of at least 95% described in
patent document WO/2015/056216, and a new
peptide SEQ. ID NO: 16. (see table 1 of
individual aminoacidic names):

| Sequence number | Peptide sequence |
|---|---|
| SEQ. ID NO: 1 | CLPCLPAASC |
| SEQ. ID NO: 2 | DCKLPCNPCA |
| SEQ. ID NO: 3 | PIYCRRTCYH |
| SEQ. ID NO: 4 | GGVCGPSPPC |
| SEQ. ID NO: 5 | VCGPSPPCIT |
| SEQ. ID NO: 6 | CGPSPPCITT |
| SEQ. ID NO: 7 | CEPAICEPSC |
| SEQ. ID NO: 8 | CVALLCRPLC |
| SEQ. ID NO: 9 | CCQSSCFKPC |
| SEQ. ID NO: 10 | SCCAPVYCCK |
| SEQ. ID NO: 11 | CCQSSCCKPSC |
| SEQ. ID NO: 12 | CGSCGCSQCSC |
| SEQ. ID NO: 13 | CQCSCCKPYCS |
| SEQ. ID NO: 14 | CQPSCCVSSCC |
| SEQ. ID NO: 15 | CVSSCCKPQCC |
| SEQ. ID NO: 16 | GGVCGPSPPCITT |

TABLE 3
List of reducing agents.
Reducing agents

| | |
|---|---|
| Peptides and | Peptide KCL (lysine-cysteine-leucine) |
| amino acids | Peptide KCCL (lysine-cysteine-cysteine-leucine) |
| | Peptide KCL-OEt (lysine-cysteine-leucine-OEt) |
| | Peptide KCCL-OEt (lysine-cysteine-cysteine-leucine-OEt) |
| | Cysteine amino acid |
| Chemicals | Dithiothreitol (DTT) |
| | Sodium bisulphite |
| | 2-mercaptoethanol |
| | Thioglycolic acid |

TABLE 4
Other components that can be used in the hair treatment composition of the present disclosure.
Adjuvant/further components

Fragrances
Adjuvant - Carbohydrates, polysaccharides, cellulose, modified cellulose, chitosan, natural
polymer derived, silicone, any mixture thereof . . .
Cationic softeners: quaternary ammonium salts, amine salts, imidazolines and quaternaries
with ester, organic oil, protein, fragrance, vitamin, emollient ester, alkanolamide, amine,
buffer, pH adjustor, salt, aliphatic alcohol, UV filter, amine oxide, chelate, fatty acid,
antimicrobial agent, antibacterial agent, PEG material, polymer, anti-static agent, alcohol, or
any mixture thereof.
Dye and pigment humectants, silicones, oils, fragrances, vitamins, buffers, antimicrobial agents,
antibacterial agents, disinfectants agents, surfactants, emulsifiers, preservatives, thickeners,
organic polymers, or any mixture thereof. anionic surfactant, amphoteric surfactant, cationic
surfactant, non-ionic surfactant. emulsifier, preservative, thickener, humectant, or any mixture
thereof
Protein
Tensioactive

TABLE 5
Ionic and eutectic components that can be used.

| Ionic liquid components (molar ratio) | Eutetic liquid components (molar ratio) |
|---|---|
| 1-butyl-3-methylimidazolium acetate with N,N- | Choline chloride-urea (1:2) |
| dimethylacetamide | |
| ([C4mim][CH3COO]/DMAc)(0.76:1 to 2.28:1) | |
| 1-Butyl-3-methylimidazolium cysteine, | Decanoic acid (DecA)- tetraoctylammonium (2:1) |
| ([C₄MIM]Cys: 1-Butyl-3-methylimidazolium | chloride (N8888-Cl) |
| hydroxide with cysteine (equimolar 20.7 mmol) | |
| (2-hydroxyethyl)trimethylammonium with amino | Malonic acid-choline chloride (1:1) |
| acid glycinate or cysteine: Cholinehydroxide (45 | |
| wt %, methanol) with amino acid (equimolar | |
| 57.79 mmol) | |
| Choline thioglycolate (thioglycolic acid 51.2 mmol: | Oxalic acid-choline chloride (1:1) |
| choline hydroxide (20 wt % in water) 256.6 mmol) | |
| 1-allyl-3-methylmidazolium dicyanamide, | Choline chloride:ethanolamine-based (1:6-10) |
| [AMIM][dca] (equimolar 0.175 mol) | |
| 1-Allyl-3-methylimidazolium chloride, [AMIM]Cl | Tryptophan fluoborate (TrpBF4)/urea (1:4) |
| (1-Methylimidazole with allyl chloride 1:1.25) | |
| 1-butyl-3-methylimidazolium chloride ionic liquid | Urea-Glucose-Citric Acid (1:1:1) |
| ([BMIM]Cl), with/without dimethyl sulfoxide | |
| (DMSO) | |
| | Urea-Glucose-Fructose (1:1:1) |
| | Urea-Tartaric Acid (1:1) |
| | Urea-Choline chloride (1:1) |
| | Glucose-Fructose-Sorbitol (1:1:1) |
| | Citric Acid-Fructose (1:1) |
| | Glucose-Citric Acid-Water (1:1:1) |
| | Tartaric Acid-Fructose (1:1) |
| | Proline-Glutamic Acid (1:1) |
| | Proline-Glutamic Acid (2:1) |
| | Proline-Oxalic Acid (1:1) |
| | Proline-Tartaric Acid (1:1) |
| | Ornitine-Tartaric Acid (1:1) |
| | Arginine-Tartaric Acid (1:1) |
| | Citrulline-Tartaric Acid (1:1) |
| | Arginine-Oxalic Acid (1:1) |
| | Proline-Malic Acid (1:1) |
| | Arginin-Malic Acid (1:1) |
| | Ornitine-Malic Acid (1:1) |
| | Citrulline-Malic Acid (1:1) |
| | Proline-Citric Acid(1-3:1) |
| | Arginine-Citric Acid (1:1) |
| | Ornitine-Citric Acid (1:1) |
| | Citrulline-Citric Acid (1:1) |
| | Proline-Glucose (1:1) |
| | Proline-Fructose (1:1) |
| | Proline-Choline Chloride (1:2-3) |
| | Choline Chloride-Malic Acid (1-3:1) |
| | Malic acid-glucose (1:1) |
| | Choline chloride-glucose (5:2) |
| | Adipic acid-choline chloride (1:1) |
| | Benzoic acid:choline chloride (2:1) |
| | Phenylacetic acid-choline chloride (2:1) |
| | Phenylpropionic acid-choline chloride (2:1) |
| | Succinic acid-choline chloride (1:1) |
| | Glycerol-choline chloride (3-2:1) |
| | Glucose-malic acid (1:1) |
| | Fructose-malic acid (1:1) |
| | Sucrose-malic acid (1:1) |
| | Glucose-citric acid (1:2) |
| | Sucrose-citric acid (1:1) |
| | Trehalose-citric acid (2:1) |
| | Thiourea choline chloride (2:1) |
| | Acetamine choline chloride (2:1) |
| | Benamide choline chloride (2:1) |

These combinations presented very promising results to achieve an environmental benign formulation to alter the shape of the human hair, from example to straighten and frizzy, and strength it. Morphological changes of human hair were presented here using the mechanism of ionic and eutectic solvents incorporating in it mixture at least one peptide: keratin based or a reducing agents (cysteine a standard amino acid with strong reducibility). Promising results were achieved (Table 6) to change the shape of human hair by the use of environmental benign solvents.

The production of the solvents can be made according with the following process:
For ionic liquids it was used the 1-Butyl-3-methylimidazolium chloride [C4mim][Cl] (20.7 mmol) with KOH (21.85 mmol) at 60° C. to form the intermediate [C4mim][OH].
For eutectic liquids it was used 1:2 molar ratio between choline chloride and urea (0.89 gr: 0.77 gr);
For auxiliary component adding at the same time 1% wt/wt of the peptide or 2% wt/wt of cysteine. The ratios prepared were loaded in a flask and were mixed at 250 rpm and 80° C. for 2 hours, to ensure the formation of a homogenous and transparent liquid.

In an embodiment, an ionic and eutectic compositions where applied during 10 minutes on Asian hair samples previously rolled on a glass road at room temperature. These results are on good way to reach the result of the chemical treatment (35% of perming) after 2 washes cycles. The perming efficiency it was calculated by the number of loops and length, before and after treatment [6].

### Example of Composition Production:

In an embodiment, it was performed several hair treatment compositions wherein
Compositing A—0.01-10% (wt/wt), preferable 1-6% (wt/wt), more preferable 1% (weight by weight)—auxiliary agent (cysteine, peptide KCL, Seq. 16-GGVCGPSPPCITT or Basic Red 2) in solution of eutectic liquid or ionic liquid, by each case as seen in FIG. 3;
   15-70° C.—Temperature of treatment;
   1 to 10 minutes—time of application.
Compositing B—20.7 mmol of ionic liquid with equimolar amounts of 1-Butyl-3-methylimidazolium chloride [C4mim] with KOH to form [C4mim][OH].
Compositing C—1 ml of a eutectic liquid: choline chloride and urea (0.89 gr: 0.77 gr)—Comparative composition;
Compositing D—An auxiliary agent: Cellulose 2-20% (wt/wt), preferably 8-12% (wt/wt), more preferably 12% (weight by weight) with co-solvent (DMSO) (up to 30%) in solution of ionic liquid based on 1-butyl-3-methylimidazolium chloride.
At room temperature—80° C.—Temperature of treatment;
The room temperature was 20° C.;
1 to 10 minutes—time of application.

In the embodiments of FIG. 3 it is shown the morphological change of human hair, Asian hair, using ionic and eutectic solvents approaches. Perming efficiency of human hair treated with those approaches, after 2 washing cycles with shampoo. (normally a chemical process of curling in similar conditions induce about 35% of perming). Pep. KP—SEQ.ID NO: 16: SGGVCGPSPPCITT.

The term “comprising” whenever used in this document is intended to indicate the presence of stated features, integers, steps, components, but not to preclude the presence or addition of one or more other features, integers, steps, components or groups thereof.

Where singular forms of elements or features are used in the specification of the claims, the plural form is also included, and vice versa, if not specifically excluded. For example, the term “a sequence” or “the sequence” also includes the plural forms “sequence” or “the sequence,” and vice versa. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

### REFERENCES

1. Gouveia, W., et al., Toxicity of ionic liquids prepared from biomaterials. Chemosphere, 2014. 104: p. 51-56.
2. Smith, E. L., A. P. Abbott, and K. S. Ryder, Deep Eutectic Solvents (DESs) and Their Applications. Chemical Reviews, 2014. 114(21): p. 11060-11082.
3. Dyer, J. M., et al., Redox proteomic evaluation of bleaching and alkali damage in human hair. International Journal of Cosmetic Science, 2013. 35(6): p. 555-561.
4. Kaur, B. J., H. Singh, and A. Lin-Greenberg, Irritant contact dermatitis complicated by deep-seated staphylococcal infection caused by a hair relaxer. Journal of the National Medical Association, 2002. 94(2): p. 121-123.
5. CAVACO PAULO, A. M., C. FREITAS DA CRUZ, and M. M. MACEDO FRANCESKO FERNANDES, PEPTIDE COMPOSITION AND USES THEREOF, in (WO/2015/056216). 2015.
6. Cruz, C. F., et al., Changing the shape of hair with keratin peptides. RSC Advances, 2017. 7(81): p. 51581-51592.

1. A hair treatment composition comprising:
a solvent; and
an auxiliary agent;
wherein the solvent is selected from the group consisting of: an ionic liquid, a eutectic mixture, a deep eutectic mixture, and combinations thereof;
wherein the auxiliary agent is selected from the group consisting of: an adjuvant, a reducing agent, a synthetic peptide with a sequence length of from 6 to 12 amino acids where 2-5 of the amino acids are cysteines, and mixtures thereof.

2. The composition of claim 1, wherein the concentration of the auxiliary agent in the composition varies from 0.01% to 10% (wt/wt).

3. (canceled)

4. The composition of claim 1, wherein the amount of solvent in the composition varies from 10 to 50,000 mmol.

5. The composition of claim 1, wherein the solvent concentration in the composition varies from 0.15 to 0.85% (wt/wt).

6. The composition of claim 1, wherein the ionic liquid is selected from of the group consisting of: 1-butyl-3-methylimidazolium acetate with N,N-dimethylacetamide; 1-Butyl-3-methylimidazolium cysteine with 1-Butyl-3-methylimidazolium hydroxide with cysteine; (2-hydroxyethyl)trimethylammonium with amino acid glycinate or cysteine and Cholinehydroxide with amino acid; Choline thioglycolate; 1-allyl-3-methylmidazolium dicyanamide; 1-Allyl-3-methylimidazolium chloride; 1-butyl-3-methylimidazolium chloride; 1-Butyl-3-methylimidazolium hydroxide; and mixtures thereof.

7. The composition of claim 1, wherein the ionic liquid is selected from of the group consisting of: 1-butyl-3-methylimidazolium chloride; 1-Butyl-3-methylimidazolium hydroxide; and mixtures thereof.

8. The composition of claim 1, wherein the eutectic mixture is selected from the group consisting of: Choline chloride-urea; Decanoic acid (DecA)-tetraoctylammonium; chloride; Malonic acid-choline chloride; Oxalic acid-choline chloride; Choline chloride: ethanolamine-based; Tryptophan fluoborate (TrpBF4)/urea; Urea-Glucose-Citric Acid; Urea-Glucose-Fructose; Urea-Tartaric Acid; Urea-Choline chloride; Glucose-Fructose-Sorbitol; Citric Acid-Fructose; Glucose-Citric Acid-Water; Tartaric Acid-Fructose; Proline-Glutamic Acid; Proline-Glutamic Acid; Proline-Oxalic Acid; Proline-Tartaric Acid; Ornitine-Tartaric Acid; Arginine-Tartaric Acid; Citrulline-Tartaric Acid; Arginine-Oxalic Acid; Proline-Malic Acid; Arginin-Malic Acid; Ornitine-Malic Acid; Citrulline-Malic Acid; Proline-Citric Acid; Arginine-Citric Acid; Ornitine-Citric Acid; Citrulline-Citric Acid; Proline-Glucose; Proline-Fructose; Proline-Choline Chloride; Choline Chloride-Malic Acid; Malic acid-glucose; Choline chloride-glucose; Adipic acid-choline chloride; Benzoic acid: choline chloride; Phenylacetic acid-choline chloride; Phenylpropionic acid-choline chloride; Succinic acid-choline chloride; Glycerol-choline chloride; Glucose-malic acid; Fructose-malic acid; Sucrose-malic acid; Glucose-citric acid; Sucrose-citric acid; Trehalose-citric acid; Thiourea choline chloride; Acetamine choline chloride; Benamide choline chloride; and mixtures thereof.

9. The composition of claim 8, wherein the eutectic mixture is choline chloride-urea.

10. The composition of claim 1, wherein the reducing agent is selected from the group consisting of: cysteine, lysine-cysteine-leucine, lysine-cysteine-cysteine-leucine, lysine-cysteine-leucine-OEt, lysine-cysteine-cysteine-leucine-OEt, dithiothreitol, sodium bisulphite, 2-mercaptoethanol, thioglycolic acid, and mixtures thereof.

11. The composition of claim 10, wherein the reducing agent is selected from the group consisting of: cysteine, lysine-cysteine-leucine, lysine-cysteine-cysteine-leucine, lysine-cysteine-leucine-OEt, lysine-cysteine-cysteine-leucine-OEt, and mixtures thereof.

12. The composition of claim 1, wherein the adjuvant is selected from the group consisting of: carbohydrate, polysaccharide, modified cellulose, cellulose, chitosan, dimethyl sulfoxide, organic polymer, humectant, oils, natural polymer derived, humectant, silicone, protein, emollient ester, alkanolamide, amine, salt, aliphatic alcohol, amine oxide, chelate, fatty acid, PEG material, polymer, alcohol, and mixtures thereof.

13. The composition of claim 12, wherein the adjuvant is selected from the group consisting of: cellulose, dimethyl sulfoxide, and mixtures thereof.

14. The composition of claim 1, wherein the synthetic peptide has a degree of identity of at least 90% with a peptide selected from the group consisting of: SEQ.ID No. 1, SEQ.ID No. 2, SEQ.ID No. 3, SEQ.ID No. 4, SEQ.ID No. 5, SEQ.ID No. 6, SEQ.ID No. 7, SEQ.ID No. 8, SEQ.ID No. 9, SEQ.ID No. 10, SEQ.ID No. 11, SEQ.ID No. 12, SEQ.ID No. 13, SEQ.ID No. 14, SEQ.ID No. 15; and SEQ.ID No. 16.

15. The composition of claim 1, wherein the synthetic peptide has a degree of identity of at least 95% with a peptide selected from the group consisting of: SEQ.ID No. 1, SEQ.ID No. 2, SEQ.ID No. 3, SEQ.ID No. 4, SEQ.ID No. 5, SEQ.ID No. 6, SEQ.ID No. 7, SEQ.ID No. 8, SEQ.ID No. 9, SEQ.ID No. 10, SEQ.ID No. 11, SEQ.ID No. 12, SEQ.ID No. 13, SEQ.ID No. 14, SEQ.ID No. 15; and SEQ.ID No. 16.

16. The composition of claim 1, wherein the synthetic peptide is selected from the group consisting of: SEQ.ID No. 1, SEQ.ID No. 2, SEQ.ID No. 3, SEQ.ID No. 4, SEQ.ID No. 5, SEQ.ID No. 6, SEQ.ID No. 7, SEQ.ID No. 8, SEQ.ID No. 9, SEQ.ID No. 10, SEQ.ID No. 11, SEQ.ID No. 12, SEQ.ID No. 13, SEQ.ID No. 14, SEQ.ID No. 15; and SEQ.ID No. 16.

17. The composition of claim 1, further comprising a softener, dye, pigment, fragrance, surfactant, emulsifier, preservative, thickener vitamin, buffer, antimicrobial agent, antibacterial agent, disinfectants agents, emulsifier, preservative, UV filter, anti-static agent, pigment, tensioactive, or mixtures thereof.

18. The composition of claim 17, wherein the surfactant is an anionic surfactant, amphoteric surfactant, cationic surfactant, or mixtures thereof.

19. The composition of claim 17, wherein the softener is a cationic softener.

20. (canceled)

21. (canceled)

22. (canceled)


SEQUENCE LISTING (16 sequences)

SEQ ID NO: 1 - protein, 10 aa, Artificial Sequence
  Description of Artificial Sequence Synthetic peptide
  75
CLPCLPAASC

SEQ ID NO: 2 - protein, 10 aa, Artificial Sequence
  Description of Artificial Sequence Synthetic peptide
  94
DCKLPCNPCA

SEQ ID NO: 3 - protein, 10 aa, Artificial Sequence
  Description of Artificial Sequence Synthetic peptide
  325
PIYCRRTCYH

SEQ ID NO: 4 - protein, 10 aa, Artificial Sequence
  Description of Artificial Sequence Synthetic peptide
  409
GGVCGPSPPC

SEQ ID NO: 5 - protein, 10 aa, Artificial Sequence
  Description of Artificial Sequence Synthetic peptide
  411
VCGPSPPCIT

SEQ ID NO: 6 - protein, 10 aa, Artificial Sequence
  Description of Artificial Sequence Synthetic peptide
  412
CGPSPPCITT

SEQ ID NO: 7 - protein, 10 aa, Artificial Sequence
  Description of Artificial Sequence Synthetic peptide
  432
CEPAICEPSC

SEQ ID NO: 8 - protein, 10 aa, Artificial Sequence
  Description of Artificial Sequence Synthetic peptide
  618
CVALLCRPLC

SEQ ID NO: 9 - protein, 10 aa, Artificial Sequence
  Description of Artificial Sequence Synthetic peptide
  717
CCQSSCFKPC

SEQ ID NO: 10 - protein, 10 aa, Artificial Sequence
  Description of Artificial Sequence Synthetic peptide
  951
SCCAPVYCCK

SEQ ID NO: 11 - protein, 11 aa, Artificial Sequence
  Description of Artificial Sequence Synthetic peptide
  1025
CCQSSCCKPSC

SEQ ID NO: 12 - protein, 11 aa, Artificial Sequence
  Description of Artificial Sequence Synthetic peptide
  1088
CGSCGCSQCSC

SEQ ID NO: 13 - protein, 11 aa, Artificial Sequence
  Description of Artificial Sequence Synthetic peptide
  1131
CQCSCCKPYCS

SEQ ID NO: 14 - protein, 11 aa, Artificial Sequence
  Description of Artificial Sequence Synthetic peptide
  1149
CQPSCCVSSCC

SEQ ID NO: 15 - protein, 11 aa, Artificial Sequence
  Description of Artificial Sequence Synthetic peptide
  1215
CVSSCCKPQCC

SEQ ID NO: 16 - protein, 13 aa, Artificial Sequence
  Description of Artificial Sequence Synthetic peptide
  SEQ ID 16
GGVCGPSPPCITT